(19) United States
(12) Patent Application Publication
(10) Pub. No.: US 2004/0022839 A1
(43) Pub. Date: Feb. 5, 2004

(54) Preparation in the form of an emulsion that contains an oxygen carrier selected from hemoglobin or hemoglobin and myoglobin, for use as a topically applicable cosmetic and for the natural regeneration of the skin in the case of oxygen deficiency

(76) Inventor: Wolfgang Barnikol, Mainz (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(21) Appl. No.: 10/333,167

(22) PCT Filed: Jun. 29, 2001
(86) PCT No.: PCT/EP01/07495

(30) Foreign Application Priority Data
Jul. 19, 2000 (DE) 10034970.6

(51) Int. Cl. A61K 9/127; A61K 38/42

(52) U.S. Cl. 424/450; 514/6

(57) ABSTRACT

The invention relates to an emulsion in the form of a cream or a lotion, and its preparation and use, which has a care effect on the skin and, at the same time, reinforces the diffusive supply of oxygen to the epidermis, in order to regenerate it and to correct an oxygen deficiency. The oxygen carrier is an untreated or a variously modified hemoglobin by itself or a hemoglobin/myoglobin mixture. Surprisingly, a diffusive oxygen supply to the skin from the outside is reinforced by means of the use of one or more oil components, together with one or more O/W emulsifiers, without the stability and diffusion of the oxygen carrier being impaired by the emulsion-forming components. Moreover, the preparation may be used as a cosmetic to achieve a natural coloring as well as an additional supply of moisture to the skin.

The invention relates to an emulsion of the claims, in the form of a cream or a lotion, which possesses a care effect on the skin and, at the same time, reinforces the diffusive supply of oxygen to the epidermis from the outside, in order to regenerate it and to correct an oxygen deficiency. The agent is rubbed into the skin. The oxygen carrier is hemoglobin alone or a hemoglobin/myoglobin mixture. It may be present in stabilized inactive or active and/or modified form. Surprisingly, a reinforced diffusive oxygen supply to the skin from the outside takes place by means of the use of one or more oil components, together with one or more O/W emulsifiers, without the stability of the oxygen carrier and its diffusion being impaired by the emulsion-forming components. Over and above this, may be used to achieve a coloring, which corresponds to normal healthy human skin color, in contrast to such color cosmetics, a natural pigment being present. The emulsion furthermore results in an additional supply of moisture to the skin. An inventive emulsion is therefore suitable as a cosmetic preparation and also, in particular, in the case of degenerative skin changes, such as after radiation, overheating (burns), ageing, not only for therapy, but also for prevention, also together with an intravasal oxygen therapy. The invention furthermore relates to a method for the production of the characterized emulsion as well as to its use, particularly also together with a gel, which contains an oxygen carrier, the oxygen carrier corresponding to the one named above.

A number of degenerative changes in the skin are caused by a chronic oxygen deficiency. Such a deficiency generally occurs if the blood circulation in the skin is no longer adequate. This happens either due to narrowing of the small arteries (the blood-supplying vessels), or if there is a vein blockage (veins are the drainage vessels of the organism; this primarily relates to the legs).

A clinician is familiar with very specific (dermatological) disease profiles in this regard, for example chronic peripheral occlusion disease with its four different stages according to FONTAINE, or diabetic angiopathy, which is caused by arteriosclerosis, or chronic vein insufficiency, that is, a malfunctioning of blood vessels.

Chronic oxygen deficiency finally results in tissue decomposition of the skin, also in the form of gangrene or ulcus cruris, so-called open leg veins, particularly frequently in the case of diabetes mellitus. If the oxygen supply is borderline, which is often the case for the elderly, even relatively short-term compression anemia, such as that, which occurs during extended bed rest, or only slight bruises of the skin result in rapid decomposition, first of the skin, and then also of the underlying tissue, which is referred to as decubitus. Obviously, subcutaneous scar formation occurs in these cases. It would be advantageous to be able to use preventive measures against this, in order to avoid the stated pathological and painful conditions prophylactically.

A detailed presentation of clinical dermatology in this regard may be found in Braun-Falco, “Dermatologie und Venerologie”, Springer-Verlag, ISBN 3-540-53542-X.

Other important problems in dermatology are skin damage after irradiation. In this case, inflammatory and degenerative symptoms are found. Here again, it may therefore be assumed that an improved diffusive oxygen supply to the skin from the outside may stop such damage; prophylactic therapy is also possible in this connection.

A third important problem is skin damage after burns; here also, a reinforced diffusive oxygen supply from the outside could help to regenerate the skin better and more quickly.

The visible external layer of the skin consists of approximately 15 layers of cells of mecrotic, that is, dying, very flat cells (keratinous cells), this layer (stratum corneum) is approximately 12 μm thick in normal skin, approximately corresponding to the diameter of round body cells. The keratinous cells are constantly shed and are formed by cell division in the so-called stratum geminativum that is located underneath them. It takes about one month for a basal cell of the stratum geminativum to be shed at the skin surface as a keratinocyte. The two layers of cells of the epidermis (stratum corneum and stratum geminativum) together have a thickness of approximately 30 μm. As a result, the conditions for oxygen diffusion are intratissue conditions, as far as the geometrical conditions are concerned; this is because the supply region of a capillary (the smallest blood vessel in the organism) has a depth of about 50 μm.

Comparative physiology shows that in terms of phylogenesis, our skin was a gas exchange organ. An earthworm, with a thickness of approximately 7 mm, absorbs its oxygen entirely by way of the skin, for example, as does a frog hibernating underwater. The fact that the human skin breathes, that is, takes in oxygen and gives off carbon dioxide, is shown by the fact that both an oxygen partial pressure and a carbon dioxide partial pressure may be measured using epicutaneous electrodes.

For the development of an agent to improve the oxygen diffusivity through the epidermis, particularly the structure of the densely packed stratum corneum must be taken into consideration. The intercellular matrix of this stratum consists of lamellary lipoid layers. arranged parallel to the surface, where very many aqueous layers alternate with lipoid double layers (see W. Umbach, Kosmetik, Thieme Verlag, ISBN 3-13-712602-9).

The aqueous layers, in particular, represent a great diffusion resistance for the oxygen flow from the outside. The agent aimed for must, however, increase the oxygen diffusivity in both types of layers. Viewed in terms of emulsion technology, the stratum comeum represents a so-called W/O form (W/O: water in oil) (see H. Mollet, A. Grubbenmann, Formulierungstechnik, Wiley-VCH, D—Weinheim).

The dermis lies below the epidermis. The former arches into the epidermis in the form of many papillae, and in every papilla there is a blood-supplying capillary with its arterial and venous end. The oxygen diffuses outward from this capillary, to the lower vital layer of the epidermis, the aforementioned stratum geminativum. The epidermis obtains the required oxygen not only from the inside, but, as has been shown (for example by Grossmann et al., Adv. Physiol. Sci. 25 (1981): Oxygen Transport to Tissue, 319-320, or by L. R. Fitzgerald, Physiol. Rev. 37 (1957), 325-336), but also, to the extent of about 50%, diffusively from the outside.

Basically, the conditions for the diffusion of the oxygen from the outside through the epidermis are more favorable than in the case of capillary delivery of oxygen. This is because, contrary to here, the oxygen partial pressure, as the driving force of the diffusion, is 150 mm of Hg in the air, not only 50 mm of Hg; furthermore, the diffusion geometry in the skin is linear not centrifugal.

In contrast to the oxygen transport that predominates within the organism by way of the lung as the diffusive oxygen, by way of convective vascular transport, and by way of diffusion from the capillary to the cells, the absorption and emission of oxygen take place close to one another in the skin; here, there is no convective oxygen transport here; the two diffusive processes, absorption and emission, are melded into one another.

Nevertheless, the basic considerations of oxygen absorption and oxygen emission are retained. The oxygen must be bound from the air with a sufficient affinity, and, on the other hand, must be driven diffusively to the vital cells of the stratum geminativum, even if the hemoglobin saturation is low, under the highest possible partial pressure of oxygen. This is achieved by the S-shaped oxygen binding of the hemoglobin if the average affinity (that is, the oxygen partial pressure at half saturation: p50 value) is adjusted suitably; the Hill index (n50 value) is a measure of the S-shape of the bonding. The stated parameters of the oxygen bonding (p50 and n50) should be adjusted optimally for the skin effect in such a way that, on the one hand, the oxygen of the air is bound completely by the hemoglobin and, on the other hand, it is also emitted once again to the cells of the stratum geminativum to a sufficient degree.

As explained, the matrix of the stratum corneum possesses a layered structure parallel to the surface, and in general corresponds to a so-called W/O emulsion. In other words, molecular hydrophilic and lipophilic layers alternate. The oxygen diffusion resistance of this matrix must therefore be reduced for the external oxygen supply. For this purpose, the diffusion resistance of both types of layers must be reduced, namely the resistance of the hydrophilic layers and of the lipophilic layers.

Until now, no fat-containing emulsions are known, on the other hand, as cosmetic products and, on the other, as external oxygen supplier, for which the oxygen is transported by means of hemoglobin or analogous natural substances, since these oxygen carriers may have a sensitive reaction particularly to emulsifiers and fats, and there is a diffusion resistance, which is also caused by mixtures of fats and emulsifiers.

One group of patents covers certain therapeutic measures that relate to the regeneration of the skin. For example, EP 275 109 A2 teaches the use of undulin, a glycoprotein, in a mixture with pro-collagen and collagen Type 1, to prevent skin ageing. The same goal is pursued in DE 19521828 A1 by the use of collagenase. WO 99/26589 A1 teaches the use of chlorophyll, cod liver oil, and camphor as active ingredients of a pharmaceutical preparation for the treatment of skin burns, sunburn, skin scalding, skin irritation, and skin abrasions.

With regard to color effects on the skin, EP 656920 B1 teaches the use of a benzopyran derivative. WO 99/11718 A1 relates to a stable external agent with very many natural plant pigments; hemoglobin is not mentioned. DE 4200349 C2 teaches the use of chlorophyll as a natural pigment, which is bound to apohemoglobin for the purpose of stabilization.

EP 706788 B1 deals with acidic dyes, which change the color of gray hair back to its original color.

Finally, EP 992236 A1 teaches how severely pigmented skin may be brightened once again, namely by the use of phospholipids, anti-oxidants, certain proteins, and certain mucopolysaccharides.

EP-B 0 673 643 describes cosmetic compositions, which contain a combination of superoxide dismutase (SOD) and a parphyrin such as chlorophyll or hemoglobin.

DE 42 36 607 relates to preparations to improve the emission of oxygen to the skin, with fluorocarbon as an essential component.

DE-PS 39 90 820 (C2) discloses light-protection agents for skin cells, based on ribonucleic acids.

JP 05 310 597 A relates to an agent for contact treatment of the skin to alleviate pain, containing an electron-accepting compound, chlorophyll or hemoglobin, for example, being used as such. The electron-accepting compound is incorporated in an electrically conducting matrix.

JP 02019311 A relates to sun screens, containing hemin or hematin, in porphoryin compounds.

JP 62111907 A describes globin-containing moisturizing, sun-screen, and erythema-inhibiting agents, blood components being inter-parried in the globin.

Lipoid emulsions are not described for these agents.

It is therefore an object of the present invention to develop an agent for increasing the external oxygen supply to the skin, with which the aforementioned oxygen deficiency diseases may be treated and prevented, wherein, at the same time, the skin is provided with special care by a sufficient supply of moisture, furthermore the oxygen carrier is not degenerated and, in addition, dyeing of the skin to the original skin color is possible. Furthermore, it shall be easy to use the agent by incorporating it into the skin.

Pursuant to the invention, this objective is accomplished owing to the fact that hemoglobin or mixtures of hemoglobin and myoglobin are incorporated in a suitable emulsion.

Pursuant to the invention, the emulsion is an oil-in-water emulsion.

Surprisingly, it was shown that penetration of the oxygen into the epidermis is possible with such an emulsion. The oil components and emulsifiers used and described below, do not bring about any any destabilization of the oxygen carrier. Rather, they supply moisture to the skin (care effect) and, at the same time, provide it with a natural color. Furthermore, the oxygen diffusion surprisingly is not hindered.

Surprisingly, the diffusion resistance of the lipophilic layers of the skin may be reduced with the oil components stated below. Pursuant to the invention, these result in an increased BUNSEN solubility coefficient (α) and thereby reduce the diffusion resistance of the lipophilic layers of the skin in accordance with FICK's First Law and because a partial pressure difference of the oxygen constantly represents the driving force for diffusion. This leads to a good effectiveness of the emulsion, since these oil components dissolve in the lipophilic layers of the skin after the emulsion is incorporated, and therefore structurally loosen these layers. Although this loosening generally results in an increased resistance, due to the increased thickness (P.Vaupel, Pflüigers Archive [Pflüiger's Archive] 361 (1976), 201-204), the diffusion resistance, pursuant to the invention, is so low overall, that the oxygen carrier may penetrate through the layers of the epidermis and supply the skin layers with oxygen.

As oil components, the inventive emulsion preferably contains components, which also serve to increase the solubility coefficient of the oxygen, as explained above, and are selected from (tri)-glycerin esters, in particular, with C4-C22 unsaturated or saturated fatty acids such as butyric acid, valeric acid, capronic acid, enath-caprylic acid, pelargonic acid, capric acid, undemayoic acid, lauric acid, tridemayoic acid, myristic acid, pentademayoic acid, palmitic acid, margarin stearic acid, nonademayoic acid and arachnic-behenic acid. In many fats, particularly natural fats, mixtures of individual fatty acids generally are present in the glycerin esters, such as mixtures of palmitinic, stearic, and oleic acids in animal fats.

Furthermore, the branched products of these and/or the unsaturated products, such as eruca, sorbic acid, linoleic acid, linolenic acid, elaeostearic acid, arachidnic acid, clupanodonic acid, or docosahexanoic acid fats, are suitable. The fats from short-chain (C4 to C8) and medium-chain (C8 to C12, particularly up to C10) unbranched or branched fatty acids such as caprylic acid and caproic acid, isobutyric acid and valerianic acid, as well as mixtures of these and mixtures of the short-chain C4 to C8 and/or the medium-chain C8 to C12, particularly up to C10 fatty acids fats, are particularly suitable.

Furthermore, pursuant to the invention, oils with an unsaturated acid component, such as oil from soybeans, aloe, apricots, plums, macadamia, roses, arnica, avocado, castor, caraway, mustard, sesame, shea, sunflowers, grape seeds, nuts, and wheat germ, are also suitable. The oils may be used alone or in a mixture or in a mixture with the fats of the aforementioned fatty acids. Fats from the aforementioned short-chain or medium-chain fatty acids or mixtures of these, particularly branched and unbranched saturated (optionally unsaturated) are preferred, and a mixture of the aforementioned oils and fats of short-chain C to C8 and/or medium-chain C8 to C12, particularly up to C10 fatty acid fats, or their mixtures, as described, are particularly preferred. A 1:1 mixture of such components is particularly preferred.

According to “Apothekenrezeptur und -defektur” (Karl Thoma, Deutscher Apothekerverlag, Stuttgart), all types of emulsion-forming agents may be used as emulsifiers. An example is unguentum emulsifimays acquosum (see DAB '97). Furthermore, possible cremophoric emulsifiers are those used for moisturizing emulsions, namely anionic, cationic, amphoteric, and nonionic emulsions, with their characteristic HLB values, namely 8-18 (HLB: hydrophilic/lipophilic balance), preferably 8-15, particularly 9-13, particularly, however, nonionic emulsifiers, such as stearates or oleates or laurates, with glycerin or glycol or polyethylene glycol or sorbitol, such as also, for example, polyethoxyylated emulsifiers, and emulsifiers known under the names “Macrogol,” Tween,” “Myrj,” or “Cremophor” with the aforementioned HLB value (for example, macrogol-9-stearate, sorbitan monostearate), or mixtures of the stated emulsifiers, see also K. Schrader, Grundlagen und Rezepturen der Kosmetika, 2^{nd} edition, Huthig-Verlag, Heidelberg, 1989, ISBN 3-7785-1491-1, pages 395-398.

Some examples of emulsifiers, which may be used pursuant to the invention, are listed in the following Table, in accordance with the Schrader reference:

| | | nonionic (n) | |
|---|---|---|---|
| | | anionic (a) | |
| Trade Name | Chemical Name | cationic (c) | HLB |
| Emulsifier 157 | Propylene glycol monostearate, | a | 7.5 |
| | self-emulsifying | | |
| G-2147 | Tetraethylene glycol mono- | n | 7.7 |
| | stearate | | |
| G-1425 | Polyethylene glycol sorbitol | n | 8.0 |
| | lanolin derivative | | |
| Acacia | USP | n | 8.0 |
| G-3608 | Polyoxypropylene stearate | n | 8.0 |
| Span 20 | Sorbitan monolaurate | n | 8.6 |
| Arlacel 20 | Sorbitan monolaurate | n | 8.6 |
| G-2111 | Polyoxyethyleneoxypropylene | n | 9.0 |
| | oleate | | |
| G-2125 | Tetraethylene glycol mono- | n | 9.4 |
| | laurate | | |
| Brij 30 1 | Polyoxyethylene lauryl ether | n | 9.5 |
| Tween 61 1 | Polyoxyethylene sorbitan | n | 9.6 |
| | monostearate | | |
| Gelatine | Pharmagel B′ | n | 9.8 |
| Tween 81 | Polyoxyethylene sorbitan | n | 10.0 |
| | monooleate | | |
| Arlypon OAG | Fatty alcohol polyglycol ether | n | 10.0 |
| G-3806 | Polyoxyethylene cetyl ether | n | 10.3 |
| Tween 65 | Polyoxyethylene sorbitan | n | 10.5 |
| | tristearate | | |
| Methylcellulose- | Methocel 15 cps | | 10.5 |
| Lamecreme SA 7 | Fatty alcohol glycol ether | n | 10.9 |
| Tween 85 | Polyoxyethylene sorbitan | n | 11.0 |
| | trioleate | | |
| G-1790 | Polyoxyethylene lanolin | n | 11.0 |
| | derivative | | |
| Myrj 45 | Polyethylene glycol mono- | n | 11.2 |
| | stearate | | |
| Arlypon 0A8 | Polyoxyethylene oleyl alcohol | | 11.3 |
| | ether | | |
| | Polyethylene glycol-400 | n | 11.4 |
| | monooleate | | |
| Cremophor S9 | Polyethylene glycol-400 | n | 11.6 |
| | monostearate | | |
| G-2161 | Polyethylene glycol-400 | n | 11.6 |
| | monostearate | | |
| Atlox 3300 | Alkylaryl sulfonate | n | 11.7 |
| Lamecreme LPM | Glycerin monodistearate | a | 12.0 |
| Atolx 3300 | Triethanolamine oleate | a | 12.0 |
| G-3910 | Polyoxyethylene oleyl ether | n | 12.2 |
| G-2127 | Polyoxyethylene monolaurate | n | 12.8 |
| Renex 690 | Polyoxyethylene alkylaryl ether | n | 13.0 |
| Lamecreme | Glycerin monodistearate | n | 13.0 |
| AOM | | | |
| Lamecreme CSM | Glycerin monodistearate | a | 13.0 |
| Lamecreme ZEM | Glycerin monodistearate | a | 13.0 |
| Renex 690 | Polyethylene glycol-400 | n | 13.1 |
| | monooleate | | |
| Tragant USP | | n | 13.2 |
| Cremophor EL | Polyoxyethylene castor oil | n | 13.3 |
| G-1284 | Polyoxyethylene castor oil | n | 13.3 |
| Tween 21 | Polyoxyethylene sorbitan | n | 13.3 |
| | monolaurate | | |
| Renex 20 | Polyoxyethylene ester mixture | n | 13.5 |
| | fatty acids and resin acids | | |
| Lamecreme | Glycerin monodistearate | a | 14.0 |
| KSM | | | |
| G-1441 | Polyoxyethylene sorbitol lanolin | N | 14.0 |
| | derivative | | |
| Lamacit 877 | Polyoxyethylene alkyiphenol | | 14.7 |
| | ether | | |
| G-7596 J | Polyoxyethylene sorbitan | n | 14.9 |
| | monolaurate | | |
| Lamacit CA | Polyoxyethylene fatty alcohol | | 14.9 |
| | ether | | |
| Lamacit GML-12 | Polyoxyethylene glycerin | n | 15.0 |
| | monolaurate | | |
| Tween 60 | Polyoxyethylene sorbitan | n | 15.0 |
| | monooleate | | |
| Myrj 49 | Polyoxyethylene monostearate | n | 15.0 |
| Cremophor 0 | Polyoxyethylene fatty alcohol | n | 16.0 |
| | ether | | |
| G-1471 | Polyoxyethylene sorbitol lanolin | n | 16.0 |
| | derivative | | |
| Cetomacrogel- | Polyethylene glycol- 100 | n | 16.1 |
| 1000 | monocetyl ether | n | 16.1 |
| Lamacit GMO- | Polyoxyethylene glycerin | n | 16.2 |
| 25 | monooleate | | |
| Lamacit GML-20 | Polyoxyethylene glycerin | n | 15.0 |
| | monolaurate | | |
| Tween 20 | Polyoxyethylene sorbitan | n | 16.7 |
| | monolaurate | | |
| Brij 35 | Polyoxyethylene lauryl ether | n | 16.9 |
| Emulgator GL- | Pentaerythritol lanolin | n | 17.0 |
| 120 | polyglycol ether | | |
| | Sodium oleate | a | 18.0 |

The emulsion furthermore contains preservatives. Substances, for example, such as dibromohexamidine, 4-dihydroacetic acid, 4-hydroxybenzoic acid, propionic acid, salicylic acid, sorbic acid, formaldehyde, paraformaldehyde, o-phenol, inorganic sulfites and bisulfites, sodium iodate, chlorobutanol, citric acid, and fumaric acid, or mixtures of these, are suitable as preservatives; in the case of the acids, their esters and salts may also be used, particularly potassium sorbate.

Substances such as sodium lactate, polylethylene glycol, sorbitol, glycerin, or pyrrolidone carboxylic acid or mixtures thereof may serve as moisture retention agents.

Preferably, methyl 4-hydroxybenzoate and propyl 4-hydroxybenzoate, are selected as preservatives, preferably in amounts of, for example, 0.02-0.25 and especially of 0.05 to 0.2%. Preferred moisture retention agents, such as sodium lactate, glycerin, propylene glycol, sorbitol, PCA (pyrrolidone carboxylic acid) are contained in an amount of 5-15%. Methyl 4-hydroxybenzoate and propyl 4-hydroxy-benzoate and glycerin, propylene glycol, and sorbitol, particularly mixtures thereof, for example, 1:1 with regard to preservatives and 1:1:1 with regard to moisture retention agents, are particularly preferred (percentages are by weight).

The diffusion resistance of the hydrophilic layers for the oxygen is reduced with the inventive formulation due to the presence of the incorporated hemoglobin or hemoglobin/myoglobin mixture by way of the mechanism of facilitated diffusion. There is a so-called increased apparent solubility of the oxygen.

The following components or mixtures thereof may be incorporated as optional substances; these are selected from circulation-promoting agents, perfume substances, substances for scar therapy/regeneration, anti-oxidants, and anti-infectious substances:

There are two possibilities for substances to promote blood circulation:

Firsly, certain essential oils are suitable for this purpose, which are then in the lipoid phase of the emulsion. These include extracts of arnica, bay, cassia, camphor, lime, marjoram, musk, nard, clove, rosemary, thyme, juniper, cinnamon, ginger, incense, red and black pepper, eucalyptus. The proportion by weight of the substances in the emulsion may amount up to 0 to 13% and preferably between 2 and 10% by weight.

Secondly, substances with a so-called strong hyperemizing substances, such as nicotinamide, nonivamide or capsaicin, come into consideration as substances, which promote blood circulation. Their proportion by weight in the emulsion may be between 0 and 3 and particularly between 0 and 1.5% weight.

Substances from each or both of the aforementioned groups may also be combined, such as nicotine and rosemary, the total amount then not exceeding 13%.

Furthermore, the emulsion may also contain anti-infectious substances (antibacterial, antiviral, and antimycotic substances). Again, certain essential oils are provided, such as suitable extracts of ajowan, jasmine, chamomile, dwarf pine, lavender, magnolia, nutmeg, ravensara, sassafras, thyme, vetiver, juniper, grapevine, lemon, tolu balsam, peppermint, myrtle, oregano, stone pine, mustard, onion, or mixtures thereof. Their proportion by weight in the emulsion may be between 0 and 13% and particularly between 2 and 10% by weight.

Furthermore, the emulsion may contain substances, once again primarily essential oils for scar treatment and regeneration. Such substances are, for example oils of angelica, geranium, salvia, rosemary, yarrow, thuja, juniperus virginiana, onion, and cedrus atlantica, or mixtures thereof.

The proportion by weight of such substances may be between 0 and 13% and particularly between 2 and 10%.

Finally, the emulsion may be protected against auto-oxidation by means of so-called anti-oxidants, particularly if it contains unsaturated fatty acids. Such substances are α-tocopherol, ascorbyl palmitate, tertiary butylhydroxy toluene (BHT), butylhydroquinone, and butylhydroxy-anisole or mixtures thereof, specifically between 0.0001 and 0.5% and preferably between 0.01 and 0.08% by weight.

Many of the substances named are also perfume materials, such as rosemary, salvia, jasmine, lavender, clove. Other substances, known for this purpose, such as musk, neroli, geranium, mandarin, or mixtures thereof, may also be used as perfume components.

The optional adjuvants of the emulsion named may be contained in the emulsion individually or combined; this also relates to the individual substances within the stated groups. The optional substances are then present in a total amount of 0-20%.

Scar treatment agents, agents to promote blood circulation and anti-infectious agents are particularly preferred, particularly when combined with one another in a ratio of 1:1:1, and are then present in a total amount of 2-20% and particularly of 5-13%.

As mentioned previously, anti-oxidants are preferably also added, if necessary, in the stated amount.

In general, a suitable emulsion contains between 20 and 90% of water, 10-80% and particularly 20-60 of oil component, 2-20% and particularly 2-10% and especially 2-5% or 3-4.5% of emulsifier, 5-15% and particularly 8-12% of moisture retention agent, and 0.02-0.025% and particularly 0.15-0.2% of preservative(s) and 0-20% of one or more of the optional additives mentioned above, as well as 0.1-3% of hemoglobin or hemoglobin/myoglobin, myoglobin may be present in amounts of 0.1-50% based on the hemoglobin.

The values given in each case are in weight percent.

With the same qualitative composition, the cream always has a lower water content than the lotion.

A water content in the emulsion, particularly of 40-80% by weight of water and 5-50% by weight of oil, results in a cream, while a water content particularly of 70-90% by weight of water and 1-20% by weight of oil results in a flowable lotion.

The ranges of the water content (cream or lotion) also depend, in particular, on the emulsifier used.

A particularly preferred formulation comprises hemoglobin or hemoglobin/myoglobin (0.1-30% and particularly 0.1-20%, based on the total weight), 5-15% moisture retention agent, 0.02-0.25% preservative, as well as 5-15% additives, selected from all the types of additives.

Preparations with 0.02-0.08 parts by weight of propyl 4-hydroxybenzoate and 0.07-0.15 parts by weight of methyl 4-hydroxybenzoate, 8-12% glycerin, propylene glycol and/or sorbitol (1:1 or 1:1:1), as well as the given amounts of oxygen carrier and oil, emulsifier and additives, are especially preferred.

It is especially preferred if hemoglobin or hemoglobin and myoglobin are present in the emulsion in an amount of 2-8%. In each case, the indicated amounts are percentages by weight.

The inventive emulsion is prepared in that water, the oil component(s), and the emulsifier(s) are are added together at room temperature, such as 22° C., or, optionally, as in the case of solid ingredients, are heated, for example to 50° and especially from 60° to 80° C., after which the additives, if present, are added, particularly after the mixture has cooled to room temperature. Finally, the hemoglobin solution or hemoglobin/myoglobin solution, which was produced separately, is added (preferably at room temperature).

It is particularly preferred if the hemoglobin in the inventive formulation is a human or bovine hemoglobin, but preferably a porcine hemoglobin, and especially one stabilized with carbon monoxide (CO). The production of such a stabilized hemoglobin is described in DE 1 970 103.7 (corresponding to U.S. Pat. No. 5,985,332). According to this document, hemoglobin/myoglobin may be transformed completely into carboxyhemoglobin/myoglobin by means of equilibration with CO; this product is stable during storage and does not have to be deligandized before further use. The modified hemoglobin may also be carbonylated.

Activation of the oxygen carrier then takes place by means of local gasification of the skin with oxygen, on which the emulsion was applied.

As mentioned, the hemoglobin may be present in a mixture with myoglobin, particularly with the latter in amounts of 0.1 to 50%, with reference to the amount of hemoglobin. Preferably, myoglobin is used in amounts of 50 to 70% hemoglobin and 50-30% myoglobin, particularly 75 to 90% hemoglobin and 25 to 10% myoglobin. The percentage information, in this connection, relates to parts by weight.

The hemoglobin or hemoglobin and myoglobin are present in the hydrophase of the emulsion in certain concentrations, as indicated, in the molecular forms as described pursuant to the invention, in molecular disperse form.

The hemoglobin used may, in particular, be human hemoglobin, porcine hemoglobin, or bovine hemoglobin. The type of myoglobin may also be selected. It may be obtained from various species of animals, such as dogs, sheep, horses, or whales.

The hemoglobins/myoglobins indicated above are known as such and are described, for example, in “Prinzipien der Biochemie” by Lehninger, Nelson, Cox (Spectrum-Verlag).

Suitable salts, which may be present during the preparation of the oxygen carrier, may be natural electrolyte components of the solution of the hemoglobin/myoglobin, such as NaCl, KCl, and NaHCO₃, particularly in the following physiological amounts (in mM): NaCl 125; KCl 4.5; NaHCO₃ 20.

Advantageously, untreated hemoglobins are used.

However, alternatively, the hemoglobins and myoglobins used may also be covalently linked, particularly with polyalkylene oxides, for the purpose of stabilization and improved tolerance, as described in U.S. Pat. Nos. 4,179,337, 5,478,805 and 5,386,014, EP 0 206 448, EP 0 67 029. This serves both for tissue tolerance and for stabilizing the products.

Covalent links of polyalkylene oxides to proteins, particularly also to (non-crosslinked) hemoglobin, are known in various forms, and described in the literature (the state of the art is described comprehensively in: Harris, J. M. (ed.): Poly (Ethylene Glycol) Chemistrv: Biotechnical and Biomedical Applications, Plenum, New York et al. 1992). In very many of these processes, bonding of the polyalkylene oxide takes place by way of a molecular bridge (“spacer”) that is formed, for example, by a bifunctional linker. Strictly speaking, in these cases a linking product of a polyalkylene oxide is linked to the protein with a linking reagent.

Preferably, those derivatives of the polyalkylene oxides, which contain a linking agent with a functional group already covalently bonded, which result in a direct chemical reaction with amino groups, alcohol groups, or sulfhydryl groups of the hemoglobins, forming covalent links of the polyalkylene oxides, are used for covalent linking of the polyalkylene oxides (polyalkylene glycols)—for example polyalkylene oxides with reactive N-hydroxysuccinimide ester groups, epoxy (glycidyl ether) groups, aldehyde groups, isocyanate groups, vinyl sulfone groups, iodoacetamide groups, imidazolyl formate groups, tresylate groups, and others. Many such monofunctionally activated polyethylene oxides are commercially available. Alternatively, non-active polyalkylene oxides may first be chemically activated, in any other suitable manner, possibly after an additional required derivativization, by means of chemical linking agents with bromocyan, a carbodiimide such as, for example, 1-ethyl-3-(3-dimethylaminopropyl) carbodiimide or N,N′-dicyclohexyl carbodiimide, cyanuric chloride (polyethylene glycols activated with this substance, 4,6-dichloro-s-triazine polyethylene glycols, are also commercially available), or other known linking agents such as, for example, 2,2′-dichlorobenzidine, p,p′-difluoro-m,m′-dinitrodiphenyl sulfone, 2,4-dichloronitrobenzene, and others (over view in Harris, J. M. (ed.): Poly (Ethylene Glycol) Chemistry: Biotechnical and Biomedical Applications, Plenum, New York et al. 1992).

Suitable polyalkylene oxides are, in particular, polyethylene glycols (polyethylene oxides), polypropylene glycols (polypropylene oxides), as well as copolymers of ethylene glycol (ethylene oxide) and propylene glycol (propylene oxide), particularly certain derivatives thereof.

As already mentioned, the bonding of polyalkylene oxides to proteins (for example: U.S. Pat. No. 4,179,337 (1979): “Non-immunogenic Polypeptides”), specifically also to hemoglobins, namely also to artificial oxygen carriers based on modified hemoglobins, is known (U.S. Pat. No. 5,478,805 (1995): “Fractionation of Polyalkylene Oxide-Conjugated Hemoglobin Solution,” U.S. Pat. No. 5,386,014 (1995): “Chemically Modified Hemoglobin as an Effective, Stable, Non-immunogenic Red Blood Cell Substitute,” EP-A 0 206 448 (1986): “Hemoglobin Combine with a Poly (Alkylene Oxide),” EP-A 0 607 029 (1982): “Oxygen Carrier”). The contents of these documents are therefore incorporated in the present document. According to the known literature, bonding of polyalkylene oxides to artificial oxygen carriers based on modified hemoglobins is undertaken only on non-crosslinked hemoglobin.

For example, the EPA 0 067 029 describes the bonding of polyalkylene glycol, for example, polyethylene/polypropylene glycol or copolymers of ethylene oxide/propylene oxide or an ether of the glycols mentioned, to a C₁-C₁₆ alcohol, an ester of the glycols mentioned with a C₂-C₁₈ carboxylic acid (preferably butyl/monostearyl ester) and an amide of glycol and a C₁-C₁₆ amine (for example, propyl-stearyl amine). As crosslinking agents, for example, N-hydroxysuccinimide, N-hydroxyphthalimide and p-nitrophenol, pentachlorophenol, are mentioned. Analogously, reactive derivatives of the polyalkylene glycol products mentioned may be used.

The molecular weight of the polymers (for example, polyethers) may be 300-20,000 and particularly 750-10,000. Molar ratios and reaction temperatures depend, in each instance, on the conditions described and known (see Examples), for example, 1-40 times excess of polyalkylene oxide/derivative, pH from 7 to 10.

Here again, hemoglobin may be bonded with effectors, as mentioned, such as, pyridoxal-5′-phosphate or pyridoxal-5′-sulfate.

EPA 0 206 448 also describes the bonding of polyalkylene oxides as mentioned above, which have an amino function and therefore are connected to hemoglobin by way of an amide bond. The molecule has the formula —CH₂—O—(CH₂)ₙ—CONHHb (n>1, particularly 1-10). In Examples 1-5, bonding, for example, with derivatized polyethylene glycol is described, for example, also when using pyridoxal-5′-phosphate hemoglobin.

U.S. Pat. Nos. 5,312,808 and 5,478,805 describe the production of hemoglobin-containing solutions with polyalkylene oxide-conjugated hemoglobin with a molecular weight in excess of 85,000 Dalton (see, in particular, Examples 1-4, in which the reaction conditions are given).

According to DE-OS 30 26 398, non-activated polyethylene glycol is reacted with 2 to 5 times the molar amount of bromocyan (pH 9-10). The residual bromocyan is removed from the reaction mixture by gel filtration, dialysis, etc., and the product is then reacted with a required amount of hemoglobin (pH 7 to 9), for example, 0.1 to 0.002 times the amount, in aqueous solution. Alternatively, polyethylene glycol is added to benzene and reacted with 2 to 5 times the molar amount of cyanic acid chloride. The reaction product, polyethylene glycol-4,5-dichloro-s-triazine, is reacted with the desired amount, for example, 1 to 0.002 moles, of hemoglobin in a buffer solution.

The methods explained above may also be used in the case of the other polymers mentioned, as well as also for myoglobin.

The hemoglobin, myoglobin may be present in untreated form (in activated or inactivated form), as mentioned. The positive factor in this connection is the sigmoid bonding characteristic of the hemoglobin, since this makes it possible to bind the oxygen from the air in large amounts, and thereby to store it, and, at the same time, to effectively give it off again diffusively, according to FICK's law, to the vital cell layers of the epidermis. If myoglobin is used in addition, this has the further advantage that its molecular weight is four times smaller than that of hemoglobin, so that an even deeper penetration of the oxygen transport molecule into the skin is possible.

The inventive formulation may be improved even further with the help of effectors of oxygen bonding. In this way, the characteristics of the oxygen bonding, as explained above (p50 and n50) may be optimized for the desired purpose. Preferably, therefore, in the preparation of the solution of the oxygen carrier, which may be untreated or modified as described, known natural effectors, such as, for example, 2,3-diphosphoglycerate, or artificial effectors such as inositol hexaphosphate or mellitic acid, are added in 1-3 times the amount, particularly in approximately equivalent amounts, reative to the hemoglobin or hemoglobin/myoglobin (Barnikol et al., Funkt. Biol. Med. 2 (1983), 245-249). Natural effectors, which do not react chemically, that is, are bound conformatively to the hemoglobin/myoglobin, are described, for example, in Lehninger et al., “Prinzipien der Biochemie” [Principles of Biochemistry], Spektrum-Verlag, 1994.

Furthermore, the hemoglobin or myoglobin described above may also, preferably also in addition to the aforementioned effectors, be chemically modified with effectors, which are covalently bonded to the hemoglobin. These include, for example, pyridoxal-5-phosphate. The synthesis of such modified hemoglobins is described in Kothe et al., Surgery 161 (1985), 563-69. Alternatively, 2-nor-2-formyl-pyridoxal-5-phosphate may also be used as an effector (van der Plas et al., Transfusion 27 (1985), 425-430). Covalently bonding effectors may be used both for hemoglobin and for myoglobin.

An oxygen carrier, produced as described below, is especially preferred for use for the inventive formulation:

Monomeric hemoglobin/myoglobin, particularly de-oxygenated, is crosslinked in aqueous electrolyte (for example, NaHCO₃, NaCl, Na lactate, or mixtures thereof) with an excess of polyalkylene oxide, for example polyethylene/polypropylene glycol (oxide), copolymers thereof or derivatives thereof, particularly an activated polyethylene glycol, such as methoxy-polyethylene glycol-N-hydroxysucciminidyl propionate (mPEG-SPA) with the desired molecular weight as described. The excess of reactant may be removed in a known manner (lysine). In this connection, an effector may preferably be linked, for example covalently, or, as described, may be added to the solution later, with a conformative effect. A hemoglobin/myoglobin produced as described above may be purified by means of chromatography (for example, by means of preparative volume exclusion chromatography), for example, by means of centrifugation, filtration, or ultrafiltration, and subsequently be processed further to yield the inventive emulsion in the manner described. If necessary, stabilization then takes place by means of carboxylation.

Alternatively, non-modified, untreated hemoglobin and myoglobin are used, which may preferably be protected against oxidation by means of carboxylation, the oxygen carrier solution having a chemically unreactive effector, as mentioned, in particular 2,3-diphosphoglycerate, in 1 to 3 times the amount, preferably in an equivalent amount relative to the hemoglobin/hemoglobin/myoglobin. Furthermore, in addition or alternatively, a hemoglobin, chemically modified with pyridoxal effectors as described by Kothe and van der Plas, may be used. For this purpose, hemoglobin is reacted with the corresponding effectors mentioned, and carboxylated, if necessary.

The use of unmodified human hemoglobin, or, in particular, of porcine hemoglobin, which is de-oxygenated pursuant to the invention and optionally carbonylated, and of corresponding de-oxygenated, unmodified myoglobin from dogs, or sheep, or horses, is particularly preferred.

Using the inventive formulation, the properties of the molecular-disperse hemoglobins are surprisingly optimized in such a way, for the most effective oxygen diffusion possible, that an advantageous oxygen supply to the skin from the outside becomes possible.

Such formulations, which contain hemoglobin/myoglobin and transport oxygen using the mechanism of facilitated diffusion, are not only of great interest for medicine, but also from the point of view of cosmetics. This is because the aging process of the skin is partly caused, to a decisive degree, also by reduced availability of oxygen for the vital, highly reactive cell layer of the epidermis. Therefore, cosmetic treatment with the inventive agent is also a medicinal treatment, and vice versa.

The inventive formulations, which contain hemoglobin or hemoglobin/myoglobin, are therefore also suitable as agents for the treatment of age-related oxygen deficiency states of the skin, in addition to treatment of oxygen deficiency states in general, or of skin symptoms caused by long-term degeneration and/or radiation or thermal effects (this includes scar formation), both preventively and therapeutically, particularly also as simultaneous co-therapy with the intravasal use of artificial oxygen carriers.

If a CO-stabilized product is used and applied to the skin, the hemoglobin or the hemoglobin and the myoglobin may be reactivated as an oxygen binder with pure oxygen, by means of short-term gasification from the outside, for approximately half an hour, but without elevated pressure, i.e. the stabilizer is removed. From then on, the dissolved artificial oxygen carrier diffusively transports oxygen more intensively, also from the air, which only contains about 20 volume percent oxygen, to a greater degree. Preferably the hemoglobin or myoglobin used is protected against oxidation before its actual use, i.e. that it is stabilized.

Alternatively, the oxygen-transporting hemoglobin/myoglobin may also be used without stabilization (oxidation protection). Although such a preparation does not keep as long as one stabilized with CO, it has the advantage that it may act directly, without prior activation with pure oxygen.

The unstabilized product is therefore more suitable for household use, while the stabilized product may be used particularly for out-patient or in-patient primary therapy.

Use of the inventive emulsion is advantageous, particularly also in combination with an intravasal oxygen therapy, the stated hemoglobins being administered as an infusion, for example, in the form of an isotonic saline solution, as the oxygen carrier.

Furthermore, it has been proven to be advantageous if the inventive emulsion, optionally in combination with an intravasal therapy as described above, takes place together with a gel, in other words before or after administration, which gel also contains the aforementioned oxygen carriers. This gel contains a solution of the oxygen carrier, particularly hemoglobin or hemoglobin and myoglobin, particularly in a solution, which contains water and, optionally, salts, and is incorporated in a formulation with a gel-like consistency, in molecular disperse form, for which purpose a gel-forming substance (0.1-20% and preferably 0.1-8%) is present. In particular, the entire solution is incorporated in a gel, such as an inorganic gel (bentonite, silicic acid) and on an organic basis, such as polyacrylic acid, gum arabic, pectin alginates, methyl cellulose, hydroethyl cellulose, starch, as well as carboxymethyl cellulose. Preferably, the gel is a hydrogel, selected from anionic polyacrylates, particularly Carbopol® of the different types, such as Carbopol 940 or 940 P. The gel is fat-free.

Natural electrolyte components of the solution of the hemoglobin/myoglobin, such as NaCl, KCl, and NaHCO₃, may be present as salts, particularly in the following physiological amounts (in mM): NaCl 125; KCl 4.5; NaHCO₃ 2.0.

The gel used also contains preservatives such as dibromohexamidine, dihydracetate acid, 4-hydroxybenzoic acid, benzoic acid, propionic acid, salicylic acid, sorbic acid, formaldehyde, paraformaldehyde, o-phenylphenol, inorganic sulfites and bisulfites, sodium iodate, chlorobutanol, and formic acid; in the case of acids, their esters and salts may also be used. The agents may be present in amounts of 0.02-0.25 or as explained below.

Preferably, preservatives are selected from among methyl 4-hydroxy-benzoate and propyl 4-hydroxy-benzoate, for example, 0.02-0.25%, particularly 0.05-0.2% and especially 0.09-0.17%. Furthermore, preferably moisture retention agents such as Na lactate, glycerin, propylene glycol, sorbitol, PCA (pyrrolidone carboxylic acid), in an amount of 5-15%, may be contained. Methyl 4-hydroxybenzoate and propyl 4-hydroxybenzoate are particularly preferred as preservatives, and glycerin, propylene glycol, and sorbitol, particularly mixtures thereof, for example, 1:1 with regard to preservatives and 1:1:1 based on the moisture retention agents, are particularly preferred as moisture retention agents. A particularly preferred inventive preparation, with a gel-like consistency, comprises hemoglobin or hemoglobin/myoglobin (approx. 0.1-30%, particularly 0.1-20%, based on the total weight), 5-15% moisture retention agent, 0.15-0.25% preservative, and 0.1 to 20%, particularly 0.1 to 8% Carbopol®. Preparations on the basis of 1-5% Carbopol®, 0.02-0.08 propyl-4-hydroxybenzoate, as well as 0.07-0.15 parts by weight methyl 4-hydroxybenzoate, 8-12% glycerin, propylene glycol and/or sorbitol (1:1 or 1:1:1) are especially preferred. Preferably, hemoglobin or hemoglobin and myoglobin is/are present in an amount of 2-8%. In each case, the amounts indicated are percent by weight and the hemoglobin and myoglobin are mixed in the ratios given above for the emulsion.

The hemoglobin in the gel is analogous to that in the emulsion, in other words untreated and/or crosslinked with polyalkylene oxide, and/or mixed with conformative and/or covalent effectors, and/or deoxygenated, and may be produced as described or also as explained below under Step I. The gel is produced in that an oxygen carrier solution as described above is incorporated in an aqueous solution, which contains the gel and the additives. Such a solution is described below under Step II, the gel being incorporated instead of the oil component and the emulsifier. In this connection, the gel may be produced in that 0.1-15 g, particularly 1-10 g of the gel-forming substance, particularly of the hydrogel, such as, for example, Carbopol®, for example 1-5 g/liter of water or aqua conservans is/are added. The additives are then added to this mixture analogously, and finally, the oxygen carrier solution, produced as described above and particularly below under Step III, is added.

The gel and the emulsion may be used one immediately after the other, in any desired order, and also with a time interval of 1 to 12 hours.

For this purpose, the two formulations are produced and packaged separately, as described, and may be made available for use, for example, in a total package. In this connection, the product is identified by name, and, optionally, by means of a different color. The gel may be marked blue or green, for example, while the cream/lotion is marked red or yellow.

The invention will now be explained in greater detail below, by means of a general procedure for producing the emulsion, as well as on by the following examples of use. The preparation takes place in three steps (I, II, and III).

Step I:

A concentrated solution of the hemoglobin or of the hemoglobin and the myoglobin, particularly the human, porcine, or bovine hemoglobin, or the bovine/sheep/equine myoglobin, which has not been modified, or also preferably has been chemically modified or provided with a chemically unreactive effector, as well as pegylated, is produced in a concentration ranging from 150 to 450, preferably from 300 to 400 g/L. For outpatient use, hemoglobin and, optionally, myoglobin are carbonylated completely by shaking with pure carbon monoxide (CO). In this connection, the solution remains in the concentration range indicated above. As mentioned, up to 50 weight percent myoglobin may be mixed with the hemoglobins.

The solution also contains between 15 and 80, preferably between 40 and 60 mM of NaHCO₃, as well as between 80 and 250, preferably 100 and 200 mM of NaCl.

In the case of the preparation of a formulation for household therapy and independent follow-up therapy, as well as for household cosmetic use, the hemoglobin/myoglobin solution carbonylated.

Step II:

The starting point is aqua conservans. This may be procured from the pharmacy or produced in-house, in accordance with NRF (Neues Rezept Formulatorium), page 6, where the composition of aqua conservans is given.

For in-house production, purified water is used, and the preservatives are added, particularly 0.02 to 0.08 propyl 4-hydroxybenzoate, preferably, however, 0.025, particularly 0.07 to 0.15 and especially 0.075 parts by weight of methyl 4-hydroxybenzoate.

Between 5% and 1515 by weight of, for example, glycerin, propylene glycol, or 70% sorbitol solution of DAB 9, preferably 8 to 12% are added as moisture retention agents, for softening the skin. Alternatively, two or three of the stated moisture retention agents may be added in the (total) amount indicated, preferably in equal parts, particularly in an amount of 8 to 12% by weight.

A suitable emulsifier is added to this solution in an amount of 2 to 20, preferably of 2-10, particularly of 2-5 or 3-4.5% by weight; furthermore fats/oils are added in amounts of 10 to 80 and preferably of 20 to 60% by weight.

Optionally, all or some of the adjuvants indicated may also be added, firstly, hyperemizing substance in an amount of 0 to 3, preferably of 0 to 1.5% by weight and secondly, essential oils to promote blood circulation, for an anti-infective effect and for regeneration (scar therapy), all in proportions by weight of between 0 and 13 and particularly between 2 and 10%. Finally, the preparation may contain anti-oxidants mentioned above in an amount of 0.0001 to 0.5, preferably of 0.01 to 0.08% by weight.

Finally, the emulsion is produced in a suitable mixer:

Step III:

The emulsion (from II) is subsequently mixed with the solution (from I) in such a way, that the hemoglobin (and myoglobin) content is between 0.1, particularly 1-30, preferably 1-20 or 1-15% by weight and particularly between 2 and 8% by weight. The emulsion may be processed once more in a mixer.

If the emulsion from Step III has a high water content, a lotion results and if it has a lower water content, a cream results. Lotion and cream have the same qualitative composition.

### EXAMPLE 1

Inventive Production of a Combined Emulsion in the Form of a Lotion

I Production of the Emulsion Base

The emulsion contains the following, per 100 parts by weight: (see also DAC.—NRF p. 25)

| | Parts by weight |
|---|---|
| Sorbitan monostearate | 2.00 parts |
| Macrogol-9-stearate | 3.00 parts |
| Glycerin 85% | 5.00 parts |
| Medium-chain triglycerides | 0.07 parts |
| Potassium sorbate | 0.14 parts |
| Purified water | 84.79 parts |

The preparation is made as follows:

In a tared 1000 mL glass bottle with a threaded neck, sorbitan monostearate, Macrogol-9-stearate, glycerin 85%, and medium-chain triglycerides are heated on a water bath until the solid components have melted. Water-free citric acid and potassium sorbate are added. The batch is filled up to 500.0 g with purified water heated to at least 60° C. The bottle is closed, shaken vigorously, and allowed to stand until it cools, while being shaken occasionally.

II Production of the Emulsion Containing the Oxygen Carrier

The emulsion base (84.0 g), produced under I, is added to 16.0 g of a 30% (by weight) porcine hemoglobin solution; the hemoglobin solution contains 50 mM of sodium bicarbonate and 150 mM of sodium chloride. The hemoglobin solution is incorporated in the emulsion base by being shaken.

### EXAMPLE 2

Inventive Production of an Oil Emulsion as a Cream

The following are placed in a vessel with stirrer:

| Oil of | | |
|---|---|---|
| apricot seed | 5.00 | g |
| wheat germ | 5.00 | g |
| aloe vera | 5.00 | g |
| macadamia | 5.00 | g |
| black caraway | 10.00 | g |
| shea butter | 35.00 | g |
| Aqua conservans | | |
| (DAC. NRF p.6) | | |
| Unguentum emulsifimays | 30.00 | g |
| (DAB 1997) | | |
| D-α-tocopherol | 0.05 | g |
| Fragrances: Essential oils | | |
| Neroli | 1 | drop |
| Geranium | 1 | drop |
| Mandarin | 1 | drop |

The componets listed are emulsified for one minute, in a single step, using a mechanical stirrer at 1,000 rpm.

### EXAMPLE 3

Production of a Gel

Methyl 4-hydroxybenzoate (1.5 g) and 0.5 g propyl 4-hydroxybenzoate were dissolved in doubly distilled water and made up to 1 L with the latter (: “DAC”).

Carbopole® 940 (5.0) g is stirred with 45 mL of glycerin and 45 mL of 1,2-propylene glycol, 850 mL of DAC are then added to the mixture and furthermore, 350 mL of doubly distilled water as well as 38 mL of 1M NaOH are added for gel formation. It was possible to mix 280 mL of a porcine hemoglobin solution with a concentration of 280 g/L homogeneously into the gel. The hemoglobin had previously been liganded to the extent of 99% with carbon monoxide.

As mentioned, cream/lotion and gel may be produced in the manner stated and then packaged together, for example, in a total package, so that both forms of administration are simultaneously available for use. In this connection, the different products are marked as mentioned, and may also be marked differently, in each instance. In particular, information about the time sequence of use may be provided.

1. A formulation, which may be applied externally, containing an oxygen carrier, wherein the oxygen carrier is incorporated in a lipoid emulsion in molecular disperse form.

2. The formulation of claim 1, wherein the oxygen carrier is selected from hemoglobin or hemoglobin and myoglobin.

3. The formulation of claims 1 or 2, wherein a total of 0.1-30% hemoglobin or hemoglobin and, of that, 0.1-50% myoglobin, based on the amount of hemoglobin, are contained.

4. The formulation of one of the claims 1 to 3, wherein the hemoglobin or myoglobin and hemoglobin is/are incorporated in an oil-in-water emulsion.

5. The formulation of one of the claims 1 to 4, wherein, in addition to the oxygen carrier, 20 to 90% of water, 10 to 80% of oil component, 2 to 20% of emulsifier, 5 to 15% of moisture retention agent, 0.02 to 0.25% of preservatives, and 0 to 20% of additives, selected from perfume substances, substances for regenerative scar therapy, for promoting blood circulation, anti-oxidants and/or anti-infectious substances are contained.

6. The formulation of claim 5, wherein, as oil component, oils with an unsaturated portion and/or fats from short-chain or medium-chain saturated branched and/or unbranched fatty acids or mixtures thereof are contained.

7. The formulation of one of the claims 5 or 6, wherein a non-ionic emulsifier with an HLB value of 8-18, particularly in pure form, or mixtures thereof, are contained.

8. The formulation of one of the claims 5 to 7, wherein the preservatives are selected from methyl 4-hydroxy benzoate and propyl 4-hydroxy benzoate, as well as from 1:1 mixtures thereof, and that the moisture retention agents are selected from glycerin, propylene glycol and sorbitol, as well as from 1:1:1 mixtures thereof.

9. The formulation of one of the claims 1 to 8, wherein the hemoglobin or myoglobin and hemoglobin is/are present, in total concentration of 0.1 to 20%, based on the total amount.

10. The formulation of one of the claims 1 to 9, wherein human, porcine, or bovine hemoglobin is incorporated as the hemoglobin and myoglobin from horses, dogs, or sheep is incorporated as the myoglobin.

11. The formulation of one of the claims 1 to 10, wherein the hemoglobin or myoglobin and hemoglobin incorporated are untreated, chemically modified and/or protected against oxidation.

12. The formulation of claim 11, wherein hemoglobin or myoglobin and hemoglobin protected against oxygen by means of carbonylation is/are incorporated.

13. The formulation of claims 11 or 12, wherein the hemoglobin or myoglobin and hemoglobin is/are covalently linked with a polyalkylene oxide and/or covalently and/or conformatively proved with a natural and/or artificial effector.

14. The formulation of one of the claims 1-13, wherein the hemoglobin or myoglobin is crosslinked with a polyalkylene oxide, particularly polyethylene oxide or polypropylene oxide or copolymers thereof.

15. The formulation of one of the claims 1-14, wherein an effector which is not chemically reactive and is selected from 2,3-diphosphoglycerate, inositol hexaphosphate, mellitic acid, or mixtures thereof, incorporated in 1-3 times the amount and particularly in an equivalent amount, based on the hemoglobin or hemoglobin/myoglobin.

16. The formulation of one of the claims 1 to 15, wherein it is present as a lotion containing 70-90% by weight of water and 1-20% by weight of oil component, the other components being present in the amounts indicated.

17. The formulation of one of the claims 1 to 16, wherein it is present in the form of a cream containing 40-80% by weight of water and 5-50% by weight of oil component, the other components being present in the amounts indicated.

18. A method for the production of a formulation containing an oxygen carrier of one of the claims 1 to 17, wherein 10 to 80% of one or more oil components, together with 2-5% of emulsifier are added with stirring to 20 to 90% of water, after which the additives, if present, are added with stirring, and finally, the oxygen carrier is added in an aqueous solution.

19. A method for the production of a formulation containing an oxygen carrier for external use, wherein a formulation of one of the claims 1 to 17, as well as a gel containing 5 to 15% of moisture retention agent, 0.02 to 0.25% of preservative, a gel-forming substance in an amount of 0.1-20%, as well as a total of 0.1-30% of hemoglobin or hemoglobin with up to 0.1-50% myoglobin, based on the total amount, are produced individually, and then packaged together for joint administration.

20. A combination preparation, which may be applied externally and is produced by the method of claim 19, comprising a preparation, which contains an oxygen carrier, in the form of an emulsion and in the form of a gel.

21. Use of a formulation of one of the claims 1 to 17, 20, or produced according to one of the claims 18 or 19, for the production of an agent for external treatment/prevention of oxygen deficiency states of the skin.

22. The use of claim 21, wherein the oxygen deficiency has been caused by degenerative and/or radiation-related as well as thermally related skin symptoms.

23. The use of a formulation of one of the claims 1 to 17, 20 or produced according to one of the claims 18 or 19, for the preparation of an agent for the treatment/prevention of age-related oxygen deficiency states of the skin.

24. The use of one of the claims 21 to 23, wherein a formulation, stabilized by means of carbonylation is used and wherein gasification of the skin with pure oxygen is carried out in order to activate the hemoglobin/myoglobin-hemoglobin as the oxygen transporter.

25. The use of one of the claims 21 to 23, wherein a unstabilized hemoglobin/hemoglobin-myoglobin formulation is used for household therapy.

26. The use of one of the claims 21 to 26, wherein a formulation of one of the claims 1 to 17, and subsequently or previously, a gel containing 5 to 15% of moisture retention agent, 0.02 to 0.25% of preservative, a gel-forming substance in an amount of 0.1-20%, as well as a total of 0.1-30% of hemoglobin or of hemoglobin and 0.1-50% myoglobin, based on the total amount, is used.

27. The use of one of the claims 21-26, wherein, in addition, an intravasal oxygen therapy with a hemoglobin oxygen carrier takes place.

28. The use of a formulation of one of the claims 1-17 as a cosmetic for a fresh skin color and a natural appearance.
